(19) United States
(12) Patent Application Publication
(10) Pub. No.: US 2002/0022262 A1
(43) Pub. Date: Feb. 21, 2002

(54) Plant tissue culture vessel

(76) Inventors: Wen-Yi Wang, Taipei Hsien (TW); Hui-Chen Hsu, Taipei City (TW)

Correspondence Address: Keith Kline, PRO-TECHTOR INTERNATIONAL SERVICES, 20775 Norada Court, Saratoga, CA, 95070-3018, US

(21) Appl. No.: 09/922,567

(22) Filed: Aug. 3, 2001

(30) Foreign Application Priority Data
Aug. 9, 2000 (TW) 089115982

(51) Int. Cl. C12M 1/24

(52) U.S. Cl. 435/304.1; 220/677

(57) ABSTRACT

The present invention relates to a type of plant tissue culture vessels which comprises a container and a cover, wherein the container is made of transparent synthetic polymers and has a opening, a stress concentration seams which be formed on the circumferential surface and/or bottom of the container and which can be focused the distribution of stresses in certain way that can be safely and easily avulsion before seedlings are removed from the culture vessel.

### FIELD OF THE INVENTION

The present invention relates to a type of plant tissue culture vessel, more particularly to a plant tissue culture vessel which can be safely and easily avulsed but not broken before seedlings are removed from the culture vessel.

### BACKGROUND OF THE INVENTION

A know plant tissue culture propagate or regenerate plant tissues under sterile conditions in suitable growth media within vessels. It has the advantages of mass-propagation, eliminating pathogens, establishing healthy stock plant production system and standardizing productions. Large amount of seedling or plant tissue organs of uniform quality can be obtained in short time for used in the horticultural or research plantation. Therefore, the successfulness of plant tissue culture influences not only the development of upstream plant biotechnology, but also the production cost of downstream agricultural plantation.

Conventionally, the culture vessels which used for plant tissue culture mass production is mainly cone-shaped glass bottle. The advantage of this type of container is that the sterile condition can be maintained easily and the cost is low. However, the disadvantages are that when seedlings are grown up and needed to be removed from the culture vessel for acclimatization or plantation, because the opening is small and the crowded seedlings are all tangled together so that it is usually difficult to remove the seedlings, and in the process can cause damages to the seedlings, reducing the survival rate and the consequence growth. This is thus a big factor for the compatibility of seedling industry. Therefore, to overcome the risk of damaging seedlings, the glass bottles are broken to remove the seedlings. Although this method reduces seedling damage, it is not easy to control the force to break the glass and broken glasses can still cause damages to seedlings and occasionally hurt operators and slows down the productivity.

### OBJECTS OF THE INVENTION

The object of the present invention is to provide a plant tissue culture vessel to overcome the above mentioned problems. Transparent synthetic polymers are molded so that seam governing the distribution of stresses is focused in certain way that when force is applied in certain places the container will split along the seam and achieve the purpose, which be avulsed the plant tissue culture vessels. It is no longer needed to break the glass bottle to remove the seedlings. This not only prevents seedlings from being damaged during the process, but also increases the survival rate and avoids unnecessary danger to the operators, and thus increases the compatibility of the industry.

### BRIEF DESCRIPTION OF THE DRAWINGS

Other features and advantages of the present invention will become apparent in the following detailed description of the preferred embodiments with reference to the accompanying drawings of which:

FIG. 1 is a schematic view, which illustrates the preferred embodiment of plant tissue culture vessel of the present invention;

FIG. 2 is a schematic view, which illustrates the container of the present invention;

FIG. 3-1 is an enlargement view of the seam of the present invention;

FIG. 3-2. is an enlargement view of the second preferred embodiment of the seam of the present invention;

FIG. 4-1. is a schematic view of the first preferred embodiment of the cover of the present invention.

FIG. 4-2. is a schematic view of the second preferred embodiment of the cover of the present invention;

FIG. 4-3. is a schematic view of the third preferred embodiment of the cover of the present invention.

### DETAILED DESCRIPTIION OF THE PREFERRED EMBODIMENT

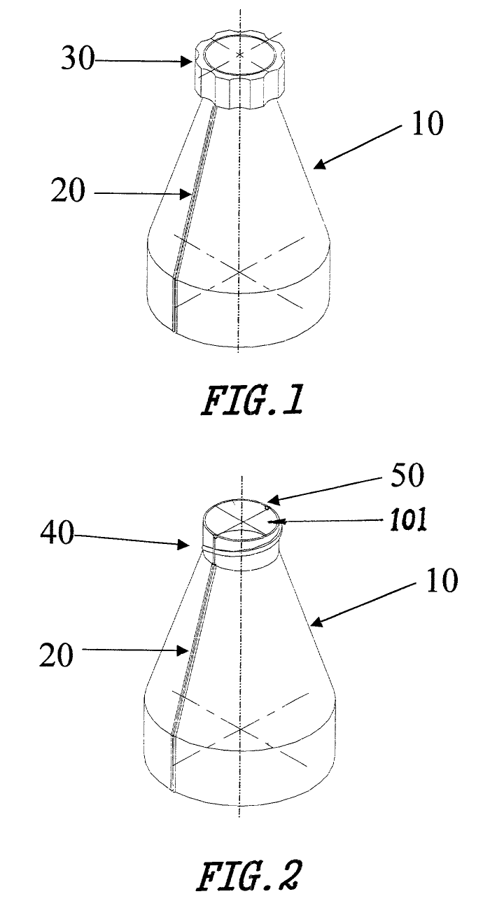

Referring to FIG. 1, the present invention provides a type of plant tissue culture container comprises a container (10) has a stress concentration seam (20) thereon, and a cover (30) can be tightly seal the container (10). Moreover, the container (10) could be colored which is added a dyes during molding process that can restrict the penetration of certain wavelengths which are between 420 nm-720 nm, in view of it has been proven that the different wavelengths could be productive of the different results during plants growth process.

As illustrated in FIG. 2, the container (10) is generally a cone-shape, which has a opening (101) on the top of the container, and the width of the opening (101) is the least wide of the container (10) even the container (10) has a neck portion thereon, a threads (40) is disposed on the outside of opening of the container (10) and can be matches the cover (30). Furthermore, The stress concentration seam (20) is continuous and mounted on the circumferential surface of the container (10)as well the bottom of the container (10) and which has a cutting site (50) that is disposed on the opening of the container (10) for avulsion. The container (10) is illustrated as cone-shape in FIG. 2, but it can be other geometrically shapes such as square or tubes.

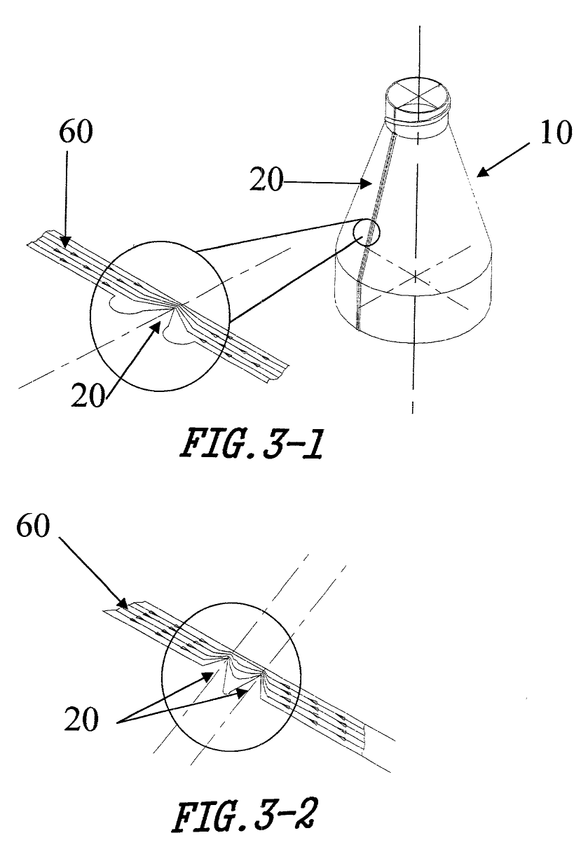

As illustrated in FIG. 3-1 is the magnified cross section of stress concentration seam (60) on the circumferential side of the container (10). It needs to be emphasized that the stress concentration seam design is different from the normal molded lines. The design of stress concentration seam is obtained through formula calculation and live test

Formula σ_{Fc}=(W−a/W)F_{ty}, wherein σ_{Fc} represents residue stress, W represents material thickness, a represents concave depth, F_{ty} represents breakdown strength; F_{ty}=σ(a/2r)_{1/2}, wherein σ represents applied stress, a represents concave depth, r=W−a;

As illustrated in FIG. 3-2 is another example of the container (10) with circumferential stress concentration seam (20).

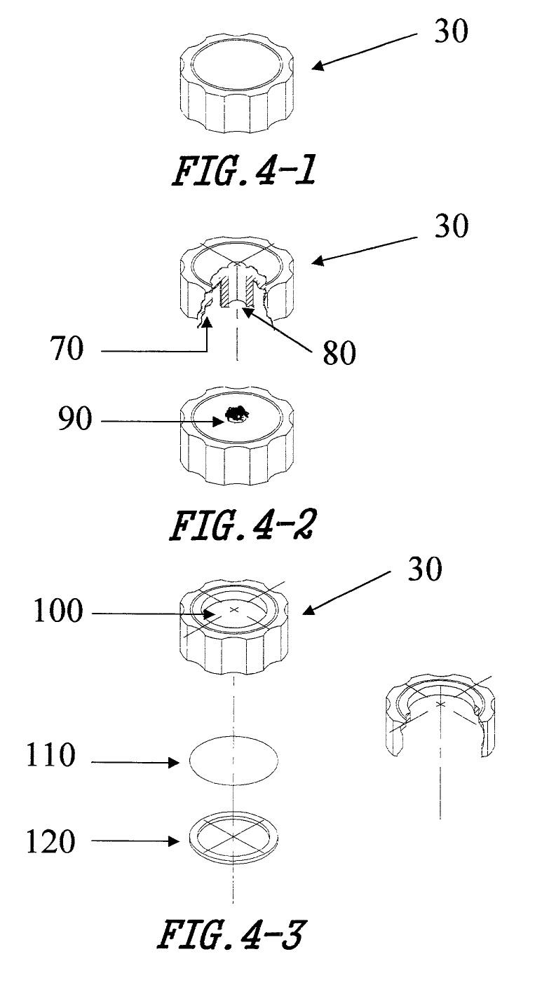

As illustrated in FIG. 4-1 is a tightly sealed container (10) with its upper cover (30) which has inner thread (70) that matches the container (40); as illustrated in FIG. 4-2 is another example of cover (30) wherein there is a ventilation hole (80) with cotton (90) for air filtration; As illustrated in FIG. 4-3 is another example of cover (30) wherein there is a ventilation hole (100) with a air filter membrane (110) sealed with a gasket (120). The air filter membrane can be replaced after being used for certain period of time for smooth ventilation.

The design of stress concentration seam can be that of FIG. 2, but can also be located on the bottom of container or be circumferential around the container or spiral around the container; in addition, there can be a cutting site on the opening of the container for applying stress to split the container apart; the stress concentration seam can be continue type as that of the FIG. 2, but can also be discontinue type with the same split apart function; the cover can be threaded onto the container, but can also be sealed with press-on; during the cultivation process, there need to have gas exchange with the outside to promote plant growth without contamination of germs. Therefore, the cover design of FIG. 4-2 and 4-3 can serve the purpose of allowing gas exchange while keeping the container sterile inside.

When the container of this invention is used for plant cultivation the design of ventilation cover can keep plant growth healthy, the stress concentration seam can make the removal of contents an easy job by simply applying proper force to the seam so that the container splits along the seam and the whole container can be open for removing seedlings. This process not only relieves the constrain of the narrow bottleneck, reduces the risk of broken glasses, shortens the labor and time, and avoids unnecessary injury to the workers so that the productivity is promoted.

While the present invention has been described in connection with what is considered the most practical and preferred embodiment, it is understood that this invention is not limited to the disclosed embodiments but is intended to cover various arrangements, included within the spirit and scope of the broadest interpretation so as to encompass all such modifications and equivalent arrangements.

We claim:

1. A type of plant tissue culture vessel, comprising:
a container has a opening and a stress concentration seam which can be split when force is applied in certain places the container will split along the seam and achieve the purpose which be avulsed the plant tissue culture vessels;
a cover can be tightly seal the container.

2. The plant tissue culture vessel as in claim 1, wherein the stress concentration seam of the container is disposed on the circumferential surface and/or bottom of the container.

3. The plant tissue culture vessel as in claim 1, wherein the width of the opening of plant tissue culture vessel is less than the inner width of the container.

4. The plant tissue culture vessel as in claim 2, wherein the stress concentration seam can extend from the circumferential surface of the container to the bottom of the container.

5. The plant tissue culture vessel as in claim 2, wherein the stress concentration seam on circumferential surface and/or bottom includes at least one.

6. The plant tissue culture vessel as in claim 2, wherein the angle which are locked between the circumferential surface of the container and the bottom of the container that is formed from zero degree to ninety degree.

7. The plant tissue culture vessel as in claim 2, wherein further comprising a cutting site, which is disposed on the circumferential surface of the stress concentration seam and near the opening end of the container.

8. The plant tissue culture vessel as in claim 2, wherein the stress concentration seam on circumferential surface and/or bottom can be continuous or discontinuous.

9. The plant tissue culture vessel as in claim 1, wherein the engagement between the cover and the container can be screw-on or press-on.

10. The plant tissue culture vessel as in claim 9, wherein further comprising a ventilation holes which is mounted on the cover.

11. The plant tissue culture vessel as in claim 10, wherein the ventilation holes can be filled with cotton or polyurethane fibers for air filtration.

12. The plant tissue culture vessel as in claim 10, wherein the ventilation holes can be equipped with air sterile filters for air filtration.

13. The plant tissue culture vessel as in claim 12, wherein the air sterile filters are replaceable.

14. The plant tissue culture vessel as in claim 1, wherein the container can be colored to restrict the penetration of certain wavelengths.

15. The plant tissue culture vessel as in claim 14, wherein the wavelength allowed to penetrating the container is between 420 nm-720 nm.
